(19) United States
(12) Patent Application Publication
(10) Pub. No.: US 2020/0237404 A1
(43) Pub. Date: Jul. 30, 2020

(54) PROTECTIVE NEEDLE CAP

(71) Applicant: Spiration, Inc. d/b/a Olympus Respiratory America, Redmond, WA (US)

(72) Inventor: Brandon J. Shuman, Kirkland, WA (US)

(21) Appl. No.: 16/262,326

(22) Filed: Jan. 30, 2019

(51) Int. Cl. A61B 17/34; A61B 1/018; A61B 10/04

(52) U.S. Cl. CPC A61B 17/3494; A61B 1/018; A61B 2017/0034; A61B 17/3478; A61B 2010/045; A61B 10/04

(57) ABSTRACT

A medical instrument having an aspiration needle and a protective needle cap. The cap protects a scope from damage due to needle insertion. The cap is placed over the end of a needle before insertion into an endoscope (e.g., bronchoscope) before use in a patient. The cap includes a first section having a first cross-sectional dimension based on an outside diameter value of a distal end of a medical device used in an endoscope and a second section having a second cross-sectional value that is smaller than the first cross-sectional value. A connection force exists between the first section and the distal end of the medical device, the connection force being greater than a frictional force between the device and an interior wall of an endoscope as the medical device is moved within the endoscope.

### BACKGROUND

The statements in this section merely provide background information related to the present disclosure and may not constitute prior art.

Needle aspiration devices, such as transbronchial needle aspiration (TBNA) devices, are used to collect samples from target tissue, such as tumors and nodules, for analysis. Bronchoscopy sampling needles have sharp tips that can penetrate the working channel of the bronchoscope when loading the needles before inserting the bronchoscope into a patient. A user may pass the long needle through the scope when the insertion tube of the scope is bent in one or more directions. When the needle tip passes through the bends in the scope, the tip can penetrate and damage the working channel of the scope.

Currently some devices include a sheath over the needle tip. When the needle is pulled back inside the sheath it is partially protected. However, the needle can still, under difficult bends of the scope, puncture through the sheath and into the working channel of the bronchoscope or the endoscope. The sheath has an open lumen for the needle to come out. This open lumen or tube doesn't flex as well as smaller diameter of the needle.

### SUMMARY

An embodiment of the application provides an exemplary device for protecting a scope from damage due to needle insertion. A cap is placed over the end of a needle before insertion into an endoscope (e.g., bronchoscope) before use in a patient.

The device includes a first section having a first cross-sectional dimension based on an outside diameter value of a distal end of a medical device used in an endoscope and a second section having a second cross-sectional value that is smaller than the first cross-sectional value. A connection force exists between the first section and the distal end of the medical device, the connection force being greater than a frictional force between the device and an interior wall of an endoscope as the medical device is moved within the endoscope.

In one aspect, at least one of the first and second sections includes a heat shrink material.

In another aspect, the second section is more flexible than at least one of a distal section of the medical device or the first section. A proximal end of the second section extends from a distal end of the first section.

In still another aspect, the second section transitions from a first cross-sectional dimension at the proximal end to a second smaller cross-sectional dimension at a distal end.

In yet another aspect, a central longitudinal axis of the distal end of the second section is parallel and separate from a central longitudinal axis of the first section when no external forces are applied to the sections.

In still yet another aspect, the device further includes a feature configured to allow removal of the device from the medical device. The feature includes one or more perforations along a longitudinal direction of at least the first section.

In further aspects, the medical device is a needle device having a needle tip at a distal end.

In still further aspects, the device is configured to be reusable.

In yet further aspects, no sheath is used for the needle, thereby allowing for more space for the needle to flex and bend around curves inside the bronchoscope.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, emphasis instead being placed upon illustrating the principles of the invention. In the drawings:

FIG. 1 illustrates a medical system formed in accordance with principles of the present invention;

FIG. 2 illustrates an x-ray view of a prior art system having a needle within a channel of a scope;

FIG. 3 illustrates a side view of a needle with a scope protection device formed in accordance with principles of the present invention; and

FIG. 4 illustrates an x-ray view of the components of FIG. 3 in a channel of an endoscope.

### DETAILED DESCRIPTION

The following description is merely exemplary in nature and is not intended to limit the present disclosure, application, or uses.

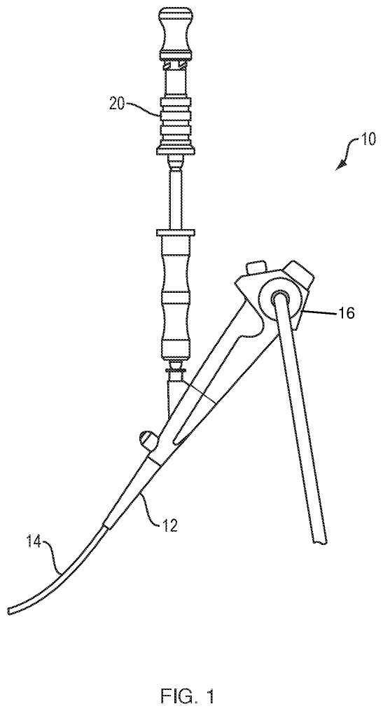

FIG. 1 illustrates a system 10 for examining, treating and/or sampling of tissue within a body. The system 10 includes an endoscope 12 with a needle aspiration device 20 received partially within the endoscope 12. A transbronchial needle aspiration (TBNA) device is an example of the needle aspiration device 20. The needle aspiration device 20 includes a handle body, a needle actuator, a stylet knob and a Luer component. The handle body is attached to a proximal end of a sheath (not shown). The needle actuator receives and is attached to a proximal end of a needle (not shown) that is slidably received within the sheath. The sheath is slidably received within a handle 16 and an insertion tube 14 of the endoscope 12.

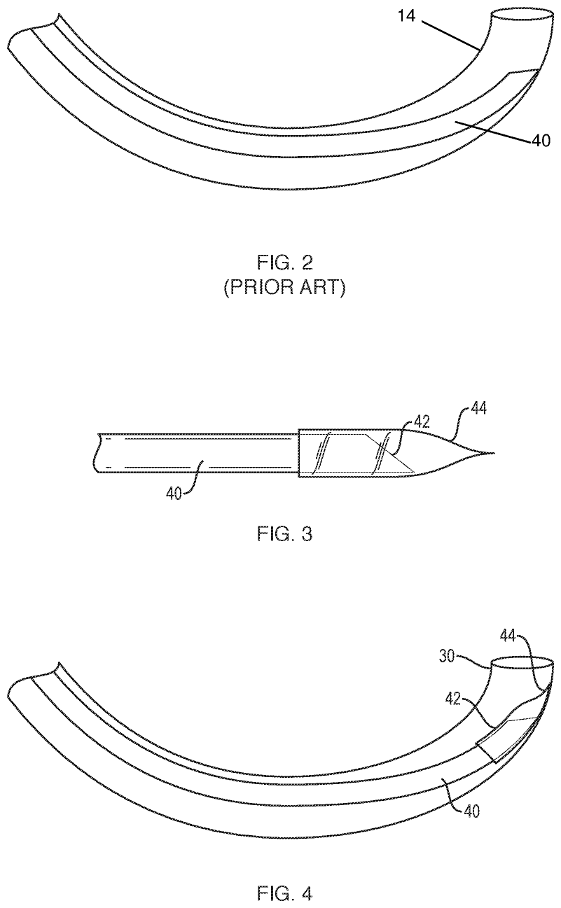

In a prior art configuration as shown in FIG. 2, before one inserts the insertion tube 14 of the endoscope 12 into a body cavity or lumen, a needle 40 of the needle aspiration device 20 is inserted into the handle 16 and the insertion tube 14. Because the distal end of the needle 40 is sharp and unprotected, the needle 40 may damage interior channels of the handle 16 and/or the insertion tube 14.

As shown in FIG. 3, a cap 44 is attached to a distal end 42 of the needle 40 or another medical device. The cap 44 includes a proximal portion that is sized to receive the distal end 42. The cap 44 includes a distal portion that has a reduced cross-sectional dimension as compared to that of the distal end 42.

As shown in FIG. 4, the distal end of the cap 44 helps guide the distal end 42 (e.g. tip) away from sharp bends in a channel 30 of the handle 16 and/or the insertion tube 14 of the endoscope 12.

In one embodiment, the distal end of the cap 44 may be more flexible in a particular direction. This may cause the cap 44 and the distal end 42 to rotate thus avoiding damage to a wall of the channel 30 by the distal end 42 of the needle 40 by allowing the tip to divert away from the wall of the channel 30. Also, the cross-sectional dimension may taper to a point that is along the longitudinal axis of the cap 44 (FIG. 4) or is offset from the longitudinal axis of the cap 44 (FIG. 4).

In one embodiment, the cap 44 includes a heat shrink material that shrinks to maintain a friction fit with the distal end 42 of the needle 40 after sufficient heat has been applied. The friction between the cap and the scope channel can be reduced by using a low friction material such as PTFE for the cap 44.

Friction between the cap 44 and the needle 40 prevents relative movement between the two. For example, heat shrink grips the sides of the needle 40 and hold it with a higher friction than any friction experienced between the scope and the cap 44. Also, the cap 44 can be designed to be thin on the sides, reducing friction with the scope, and thicker in front of the needle tip. This would prevent the needle from penetrating through the tip in the axial direction.

In one embodiment, the proximal portion of cap 44 is configured to snap fit onto the distal end 42 of the needle 40. The cap 44 may include internal barbs (not shown) or other features that keep the proximal portion of cap 44 in place while the needle 40 is passed through the endoscope 12. The cap 44 may be made of Polycarbonate, Polyurethane, PTFE or comparable biocompatible materials. In one embodiment, a main shaft of the cap 44 could have a higher durometer around the needle shaft and a lower durometer proximal to the tip.

After the needle 40 has been inserted into the endoscope 12 and before the endoscope 12 is inserted into a body cavity/lumen, the distal end 42 of the needle 40 with the cap 44 is advanced beyond the end of the insertion tube 14. The cap 44 is then removed and the distal end 42 of the needle 40 is retracted inside the insertion tube 14. Because the cap 44 has not made contact with any biological material, the cap 44 may be reused on subsequent sterile devices.

In one embodiment, removal of the cap 44 may occur a few different ways. For example, the cap 44 could be skived off with a blade. Or, just the act of pulling on the distal tip would elongate the cap 44, thus making it thinner and causing it to rip. A tearing “rib” or serrated line (i.e., peeling strip) in the cap 44 could be designed so it tears along the side, making it easy to pull off by the user.

### Embodiments

A. A device comprising: a first section having a first cross-sectional dimension based on an outside diameter value of a distal end of a medical device used in an endoscope; and a second section having a second cross-sectional value that is smaller than the first cross-sectional value, wherein a connection force exists between the first section and the distal end of the medical device, the connection force being greater than a frictional force between the device and an interior wall of an endoscope as the medical device is moved within the endoscope.

B. The device of A, wherein at least one of the first and second sections comprises a heat shrink material.

C. The device of A or B, wherein the second section is more flexible than at least one of a distal section of the medical device or the first section.

D. The device of A, B or C, wherein a proximal end of the second section extends from a distal end of the first section.

E. The device of D, wherein the second section transitions from a first cross-sectional dimension at the proximal end to a second smaller cross-sectional dimension at a distal end.

F. The device of E, wherein a central longitudinal axis of the distal end of the second section is parallel and separate from a central longitudinal axis of the first section when no external forces are applied to the sections.

G. The device of any of A-F, further comprising a feature configured to allow removal of the device from the medical device.

H. The device of G, wherein the feature comprises one or more perforations along a longitudinal direction of at least the first section.

I. The device of any of A-H, wherein the medical device is a needle device having a needle tip at a distal end.

J. The device of any of A-I, wherein the device is configured to be reusable.

K. A needle device comprising: a flexible needle comprising: a sharp distal tip; and a flexible shaft attached to a proximal end of the distal tip; and a cap configured to protect an endoscope from the sharp distal tip of the flexible needle comprising: a first section having a first cross-sectional value based on an outside diameter value of the flexible needle; and a second section having a second cross-sectional value that is smaller than the first cross-sectional value, wherein a connection force exists between the first section and the flexible needle, the connection force being greater than a frictional force between the cap and an interior wall of an endoscope as the flexible needle is moved within the endoscope.

L. The needle device of K, wherein at least one of the first and second sections comprises a heat shrink material.

M. The needle device of K or L, wherein the second section is more flexible than at least one of a distal section of the flexible needle or the first section

N. The needle device of any of K-M, wherein a proximal end of the second section extends from a distal end of the first section.

O. The needle device of N, wherein the second section transitions from a first cross-sectional dimension at the proximal end to a second smaller cross-sectional dimension at a distal end.

P. The needle device of O, wherein a central longitudinal axis of the distal end of the second section is parallel and separate from a central longitudinal axis of the first section when no external forces are applied to the sections.

Q. The needle device of any of K-P, wherein the cap further comprises a feature configured to allow removal of the cap from the flexible needle.

R. The needle device of Q, wherein the feature comprises one or more perforations along a longitudinal direction of at least the first section.

S. The needle device of any of K-R, wherein the cap is configured to be reusable.

The description of the invention is merely exemplary in nature and variations that do not depart from the gist of the invention are intended to be within the scope of the invention. Such variations are not to be regarded as a departure from the spirit and scope of the invention.

What is claimed is:

1. A device comprising:
a first section having a first cross-sectional dimension based on an outside diameter value of a distal end of a medical device used in an endoscope; and
a second section having a second cross-sectional value that is smaller than the first cross-sectional value,
wherein a connection force exists between the first section and the distal end of the medical device, the connection force being greater than a frictional force between the device and an interior wall of an endoscope as the medical device is moved within the endoscope.

2. The device of claim 1, wherein at least one of the first and second sections comprises a heat shrink material.

3. The device of claim 1, wherein the second section is more flexible than at least one of a distal section of the medical device or the first section.

4. The device of claim 1, wherein a proximal end of the second section extends from a distal end of the first section.

5. The device of claim 4, wherein the second section transitions from a first cross-sectional dimension at the proximal end to a second smaller cross-sectional dimension at a distal end.

6. The device of claim 5, wherein a central longitudinal axis of the distal end of the second section is parallel and separate from a central longitudinal axis of the first section when no external forces are applied to the sections.

7. The device of claim 1, further comprising a feature configured to allow removal of the device from the medical device.

8. The device of claim 7, wherein the feature comprises one or more perforations along a longitudinal direction of at least the first section.

9. The device of claim 1, wherein the medical device is a needle device having a needle tip at a distal end.

10. The device of claim 1, wherein the device is configured to be reusable.

11. A needle device comprising:
a flexible needle comprising:
a sharp distal tip; and
a flexible shaft attached to a proximal end of the distal tip; and
a cap configured to protect an endoscope from the sharp distal tip of the flexible needle comprising:
a first section having a first cross-sectional value based on an outside diameter value of the flexible needle; and
a second section having a second cross-sectional value that is smaller than the first cross-sectional value,
wherein a connection force exists between the first section and the flexible needle, the connection force being greater than a frictional force between the cap and an interior wall of an endoscope as the flexible needle is moved within the endoscope.

12. The needle device of claim 11, wherein at least one of the first and second sections comprises a heat shrink material.

13. The needle device of claim 11, wherein the second section is more flexible than at least one of a distal section of the flexible needle or the first section

14. The needle device of claim 11, wherein a proximal end of the second section extends from a distal end of the first section.

15. The needle device of claim 14, wherein the second section transitions from a first cross-sectional dimension at the proximal end to a second smaller cross-sectional dimension at a distal end.

16. The needle device of claim 15, wherein a central longitudinal axis of the distal end of the second section is parallel and separate from a central longitudinal axis of the first section when no external forces are applied to the sections.

17. The needle device of claim 11, wherein the cap further comprises a feature configured to allow removal of the cap from the flexible needle.

18. The needle device of claim 17, wherein the feature comprises one or more perforations along a longitudinal direction of at least the first section.

19. The needle device of claim 11, wherein the cap is configured to be reusable.
